(19) United States
(12) Patent Application Publication
(10) Pub. No.: US 2008/0300508 A1
(43) Pub. Date: Dec. 4, 2008

(54) Diagnostic patch

(75) Inventor: Keren Tomer, (US)

Correspondence Address: DON J. PELTO;Sheppard, Mullin, Richter & Hampton LLP, 1300 I STREET, NW, 11TH FLOOR EAST, WASHINGTON, DC, 20005, US

(73) Assignee: Inverness Medical Switzerland GmbH

(21) Appl. No.: 11/806,170

(22) Filed: May 30, 2007

(51) Int. Cl. A61B 5/157

(52) U.S. Cl. 600/583

(57) ABSTRACT

The Present invention generally relates to medical diagnostics devices and more specifically to a diagnostic patch attachable to a subject's skin for performing a rapid blood test.

A well-known format for performing assays where a sample is applied to a test strip impregnated with assay specific reagents.

### BACKGROUND OF THE INVENTION

1. Field of the Invention

The present invention generally relates to medical diagnostics devices and more specifically to a diagnostic patch attachable to a subject's skin for performing a rapid blood test.

2. Discussion of the Related Art

Diagnostics test kits for rapid detection of specific medical conditions and diseases are becoming increasingly widespread in the field of medical diagnosis. Such kits allow for immediate point-of-care diagnosis in the most basic of healthcare settings with no need for expensive instrumentation and with minimal specialized training.

A well-known format for performing rapid assays is the lateral flow platform where a sample is applied to a test strip impregnated with assay specific reagents, typically a binding assay such as immunoassay. The sample is applied to one end the test strip and is drawn through the strip by capillary action to pass through a reaction zone where the analyte, when present, reacts with the pre-impregnated reagents and further into a detection zone where the appearance of a visible or otherwise detectable signal indicates presence of the analyte in the sample. There exist many variations of this basic structure, regarding the number and nature of the immobilized, labeling and other reagents located along the strip and their interaction with the analyte, as well as to the nature and formation of the signal. A great variety of analytes may be detected in this manner. In particular relevant to the present invention are rapid diagnostic blood tests where the presence of a specific substance in the blood is indicative of the presence or absence of a disease or a physiological condition, such as for example, the Determine® series from Inverness Medical for the detection of sexually transmitted diseases, including HIV, Hepatitis B and Syphilis.

Although available rapid blood testing kits, such as the Determine® series, significantly shorten and simplify blood test procedures, they still require separate actions for collecting a blood sample from a tested subject and transferring the sample to the test device for analysis. Collection of blood sample usually involved withdrawing blood by means of a syringe or the use of a lancet to injure a body area such as a fingertip and collecting blood from the injury by means of a capillary tube. Such procedures are typically performed by a trained person and may expose the person to infectious blood samples. Moreover, blood sample collection and sample testing are not necessarily performed by the same person. Often blood specimens are collected in one location while tests are performed in another location. This requires transporting the collected specimens and a double identification recordation first for labeling the collected blood samples, then for labeling the test devices, e.g., test strips. In particular, where large groups of people are to be screened for an infectious agent, such as for example HIV, and where it is possible that tested individuals will not come for follow-up, it is particularly desirable to have means for obtaining rapid results while providing easy identification means that prevents possible mismatch between test subjects and test results.

It will be therefore desirable to have an all-in-one self-contained diagnostic device, which allows performing both collection and subsequent analysis of a blood sample in the same device with no need to transfer the collected sample to a separate test device. Such a device will simplify test procedure, will reduce the time required for the test and will minimize exposure of personnel to collected blood samples. It will be also desirable to have such an all-in-one blood test device which is attachable to the subject being tested, so that it can be attached to the subject just before the test is started and remains thereon until the test is complete. Such a device will ensure that test results are made known to the tested subject. It further eliminates the need to manage separate identification labels for blood samples and for test devices and prevents possible mismatch between tested subjects and test results.

### SUMMARY OF THE PRESENT INVENTION

Accordingly it is a general object of the present invention to provide an all-in-one self-contained rapid diagnostic device for collecting and analyzing a blood sample of a test subject by a one-step operation with no need to manipulate blood samples.

It is a further object of the invention to provide such a diagnostic device as defined above which is configured as a flexible patch that can be easily attached to the skin of the test subject to be left thereon until the test is complete and test results are visibly displayed.

Such a device has the advantages of simplifying test procedure and minimizing exposure of health care practitioners to blood samples and lancing devices. It has the further advantage of eliminating the need to manage separate identification records of blood samples and test devices.

Accordingly the present invention provides diagnostic device attachable to a skin segment of a test subject for a rapid detection of a pre-selected analyte in the subject's blood. The analyte may be a blood borne pathogen or any other substance the presence of which is indicative of a disease or a physiological condition.

The diagnostic device of the invention comprises a flexible patch attachable to the subject's skin, a puncturing unit for producing a blood sample of the subject, and an at least one test element embedded in said patch in a flow communication with the blood sample. The diagnostic device further comprises a display window through which test results can be visibly read. The puncturing unit may comprise a lancing element mounted within a flexible housing but may be any other lancet unit with an automatic retraction mechanism.

According to a preferred embodiment the invention, the flexible patch comprises a first flexible sheet comprising an aperture located opposite the puncturing unit and a second flexible sheet overlaying said first flexible sheet wherein said test element is sandwiched between the first and the second flexible sheets in a fluid communication with the aperture.

The test element is preferably a diagnostic strip adapted for a lateral flow assay of a whole blood sample wherein the assay may be an immunoassay, an enzymatic assay, a biochemical assay or a chemical assay. Preferably said assay is a positive/negative assay for detecting the presence of an analyte in the blood sample. Yet, according to other embodiments, the assay may be a quantitative or a semi-quantitative assay for detecting the concentration of the analyte. In accordance with a certain embodiment of the invention the analyte is a blood born pathogen. Preferably the diagnostic strip comprises a sample receiving zone, a whole blood separation zone for entrapping and retaining red blood cells, a reaction zone and a detection zone.

Optionally the device further include a reservoir of a releasable reagent solution adapted to release the reagent solution to facilitate running the diagnostic test. The reservoir may comprise a blister made of liquid impermeable film for encapsulating the reagent solution. The blister may be accommodated within the same puncturing unit that produces the blood sample or within a second puncturing unit located upstream to the blood sample producing unit. Optionally the device may further comprise a safety means for preventing premature activation of the puncturing units.

### BRIEF DESCRIPTION OF THE DRAWINGS

The present invention will be understood and appreciated more fully from the following detailed description taken in conjunction with the drawings in which:

FIG. 1 illustrates a diagnostic patch of the present invention attached to the arm of a test subject;

FIG. 2 is a plane top view of a diagnostic patch of the invention;

FIG. 3 is a plane bottom view of a diagnostic patch of the invention;

FIG. 4 is an exploded isometric view of a diagnostic patch in accordance with a preferred embodiment the present invention;

FIG. 5 is an exploded longitudinal cross section of a diagnostic patch illustrating the multiplayer structure of the patch;

FIGS. 6A to 6C illustrate different possible arrangements of forming flow communication between the test strip and the blood sample;

FIG. 7 illustrates a typical test strip suitable for use in the present invention;

FIGS. 8A and 8B illustrate two embodiments of a puncturing unit with a safety means for preventing a premature puncturing;

FIGS. 9A and 9B are partial views of two configurations of a patch of the invention incorporating a reservoir of reagent solution.

### DETAILED DESCRIPTION OF THE PREFERRED EMBODIMENT

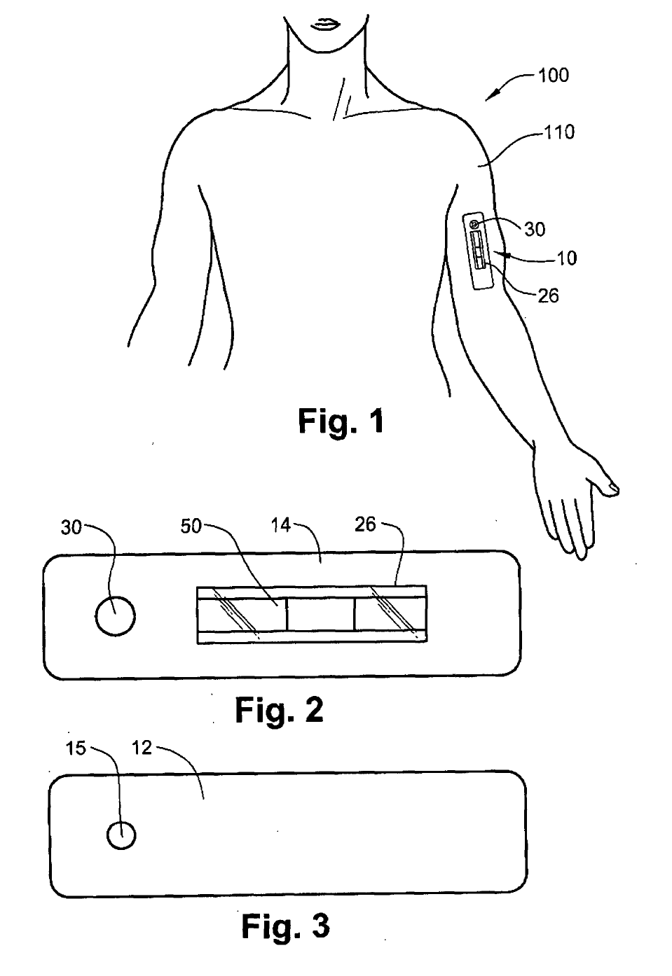

The present invention provides an all-in-one self-contained blood-test diagnostic device attachable to the skin of a tested subject. The device allows for performing a blood test by one operation step with no need to handle or manipulate blood samples, thus preventing exposure of health care providers to blood samples. The device has the further advantage of reducing to zero the time lag between initial sample collection and test performing. The device is configured as an adhesive skin patch that is easily adhered to a skin segment of the tested subject such as in an arm as depicted in FIG. 1, a forearm, a thigh or the like, and preferably remains thereon until test results are displayed. The patch includes a puncturing unit for producing a blood sample, at least one test element in flow communication with the blood sample so produced and a display window for viewing the test results. The test element may be any test element known in the art for rapid detection of an analyte in a whole blood sample. Preferably the test element is a lateral flow diagnostic strip configured for displaying test results within less than 30 minutes, more preferably within 5 to 15 minutes, by a clearly interpreted visible signal with no need for further equipment for interpretation. By selecting a suitable test element, the diagnostic patch of the invention may be used to detect various disease and medical conditions. For example, the patch may be used for a rapid detection of infectious diseases such as AIDS and hepatitis or for diagnosing myocardial infarction by monitoring cardiac markers.

The device of the invention allows for screening a large number of subjects in a simple straightforward manner with no need to manage separate labeling for samples and for test devices. The patch may be used in developing areas where it is necessary to screen population for infectious diseases or for screening admitted patients in emergency rooms. The patch may also be used at blood donation sites for diagnosing potential donors.

The term analyte as used throughout the application is meant to denote any substance, compound or composition to be detected in a blood sample, including antigens, antibodies, hormones, proteins, enzymes, nucleic acids, drugs, or any other natural or synthetic substance whose presence and/or concentration in the blood may be of interest.

Referring to FIGS. 1-5, where like numerals refer to like components, there is shown a diagnostic patch, generally designated 10, in accordance with one preferred embodiment of the present invention. As illustrated in FIG. 1, patch 10 is adapted to be adhered to the skin of a test subject 100, for example to the skin of arm 110. Patch 10 includes a puncturing unit 30 accessible from the top surface of the patch and a display window 26 through which test results are visibly displayed. The patch may be adhered to any suitable body area of the test subject by means of adhesive face 12. After being attached to the test subject, puncturing unit 30 is activated to puncture the dermal tissue underneath for creating an opening therein through which subject's blood is released. The blood travels to a rapid diagnostic test device 50, embedded within the patch, pre-selected for performing the desired diagnostic test. Upon completion of the test, results are visibly displayed through window 26.

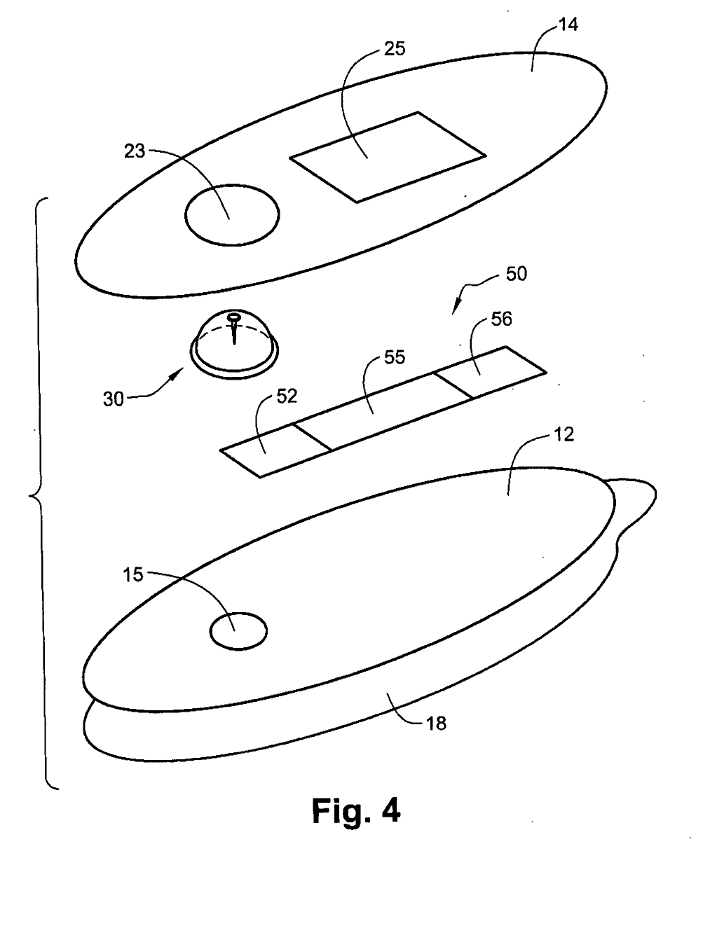

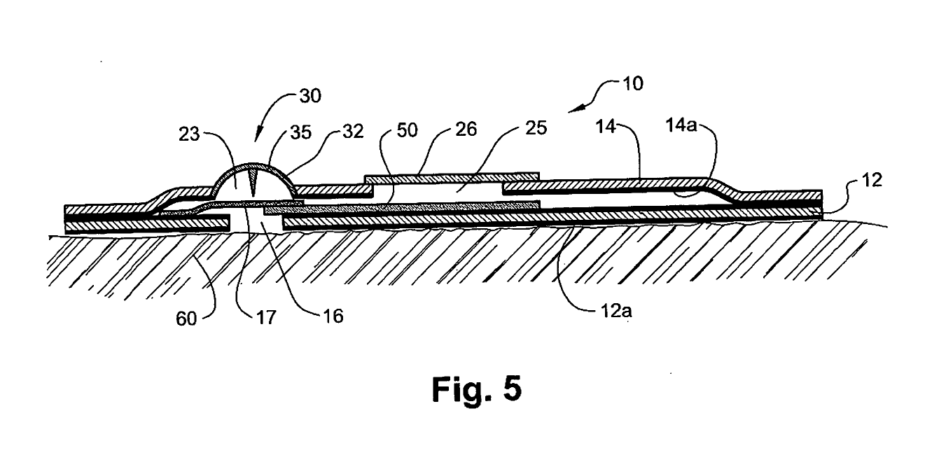

As best seen in FIGS. 4 and 5, patch 10 is constructed from a first flexible sheet 12 cut to include an aperture 15 which extends between the two opposite faces of the sheet and is covered by a liquid-impermeable film, a second flexible sheet 14 overlaying sheet 12, a diagnostic strip 50 sandwiched between sheets 12 and 14, and a puncturing unit 30 located above aperture 15. In accordance with the embodiment shown here, test strip 50 is disposed substantially along the longitudinal axis of patch 10 having its sample receiving end 52 extending over and into aperture 15 underneath film 17. When patch 10 is adhered to skin 60, a cavity 16 is formed between the skin and film 17. Top sheet 14 is cut to include an elongated opening 25 aligned with detection zone 55 of strip 50 and a second opening 23 for receiving puncturing unit 30. Yet according to other embodiments puncturing unit 30 may be placed on top sheet 14, in which case lancing element 35 may be adapted to penetrate through sheet 14. Patch 10 may further include a transparent film 26 slightly larger than opening 25 for protecting test element 50. Film 26 may be placed over top sheet 14 as depicted in FIG. 5, or alternatively may be placed over strip 50 between sheets 12 and 14. Optionally, patch 10 may further include an opaque cover (not shown) removably attached over window 25 so as not to expose the test results to passersby and to keep the subject privacy. Such a cover may be easily removed when the test is complete. After being assembled, patch 10 may be sterilized and enclosed in a sealed package (not shown) to be ready for use.

In the embodiments shown here, patch 10 is essentially of a rectangular or oval shape, however it will be easily realized that patch 10 may assume other shapes without departing from the scope of the invention, as long as it is dimensioned to accommodate all essential components. It will be also realized that the layers of material used in the construction of patch 10 are quite thin and that the thickness of the different layers in FIG. 5 is exaggerated for the clarity of illustration. Thus, although an impression is formed as if overlapping areas between various layers are bulky and irregular, in reality, such overlapping areas are substantially flat so that the overall shape of the patch is generally of a flat flexible sheet with the exception of puncturing unit 30, which protrudes above the upper surface of the patch. Due to its flexibility, patch 10 can be easily attached to curved surfaces and may be applied to various body areas.

Flexible sheets 12 and 14 may be made from a large variety of nonirritating, hypoallergenic, cloth or plastic materials known in the art for use in the fabrication of skin adhesive tapes and bandages. The sheets may be selected from ready-made available tapes or may be especially designed and fabricated for the present purpose. Sheets 12 and 14 may also include multiple perforations as known in the art for preventing building up of sweat underneath the patch. Preferably, flexible sheet 12 is a double-sided adhesive tape having both faces coated by layers 12a, 12b of pressure sensitive adhesive. Top sheet 14 is preferably a one-sided adhesive tape coated with pressure sensitive adhesive layer 14a. However, it will be easily realized that other arrangements are possible that allow for overlaying the various layers of patch 10. A release liner 18 made from any of a variety of materials known in the art protects the adhesive coating 12a of patch 10 until the patch is to be used. Release liner 18 may have a similar size and shape as patch 10 or may be slightly larger for creating a rim or a tab to facilitate its removal. Similarly, release liner 18 may be divided into two or more abutting or partially overlapping parts for facilitating the attachment of patch 10 to skin.

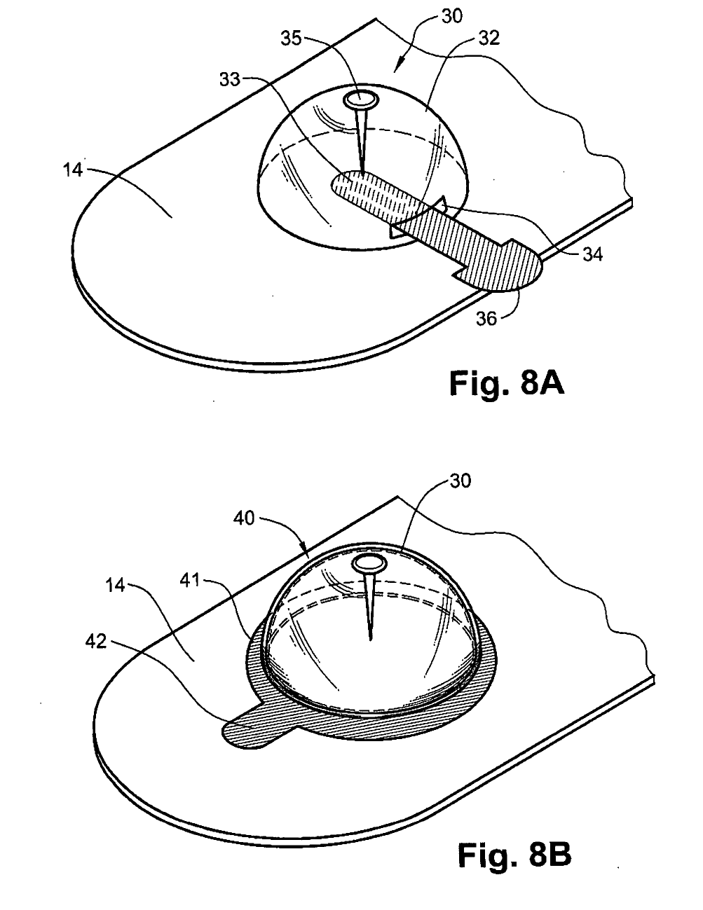

Puncturing unit 30, comprising a housing 32 and a sterile lancing element 35, is configured for penetrating the skin for drawing blood. Lancing element 35 may be a hollow or a solid needle or any other sharp sterile element suitable for lancing dermal tissue for producing at least one drop of blood. The size and shape of lancing element 35 as well as the depth to which it penetrates the skin tissue may vary and designed in accordance with the amount of blood required for performing the test and the density of blood vessels in the skin tissue in the body area to which the diagnostic patch is applied. In its default position lancet 35 is having its pricking tip suspended above the upper surface of patch, substantially pointing at the center opening 15. Upon pressing, lancing element 35 penetrates through layers of patch 10, if present, and further into the underlying dermal tissue 60 to create an opening therein. Puncturing unit 30 is provided with a return/retract mechanism for withdrawing lancet 35 back to its default position once the puncturing unit is released. Various mechanisms may be employed for the firing of unit 30. In accordance with some embodiments, lancing element 35 may be fixedly mounted within an elastic or spring-loaded capsule-like housing 32 such that upon pressing, lancet 35 is pushed down to puncture skin while upon release, housing 32 as well as the lancet bounce back to their default position. Alternatively, lancet 35 may be movably mounted within a rigid housing to enable movement of element 35 relative the housing. For example, element 35 may be mounted to housing 32 by a spring loaded mechanism, or by another biasing mechanism, that biases element 35 to its default position above the upper surface of patch 10 and provided with a trigger element accessible from outside the housing that allow for firing the unit. Unit 30 may be further provided with a safely locking means for preventing unintentional premature actuation of the unit. FIGS. 8A and 8B depict two possible configurations of such a safety means. In accordance with the configuration shown in FIG. 8A, such a safety means is formed by a movable rigid plastic slip 33 that extends across opening 23 of unit 30 and is having an outward extension 36 extending out of housing 32 through opening 34. In its locking position slip 33 is placed beneath lancet 35 to block the lancet movement. Upon pulling slip 33, the lancet is ready for activation. A second safety mechanism is depicted in FIG. 8B according to which a removable rigid cap 40, attached to layer 14 by means of adhesive rim 41, is placed on top of unit 30 protecting unit 30 from being activated. A pulling tab 42 extending from rim 41 allows for removing protective cap 40 immediately before the test is to be performed.

Upon being punctured, an opening is formed in dermal tissue 60 through which at least one drop of blood is generated in cavity 16 on the surface of the skin. Film 17, made of a liquid repellent, non-absorbent material, such as polyethylene, serves as a liquid barrier that prevents blood generated in cavity 16 from flowing or diffusing out of the cavity except via test strip 50. For the same reason, the edges of aperture 15 may be impregnated with a liquid repellent material. It will be appreciated that the shape of aperture 15 shape is not limited to a circle and that the aperture may be assume other forms to facilitate the guidance of blood into the receiving zone 52 of strip 50. Further in order to facilitate blood migration from aperture 50 to strip 50, patch 10 is preferably adhered to the skin in a vertical orientation with puncturing unit 30 at the upper end so that blood transport in the desired direction is enhanced by gravitation force.

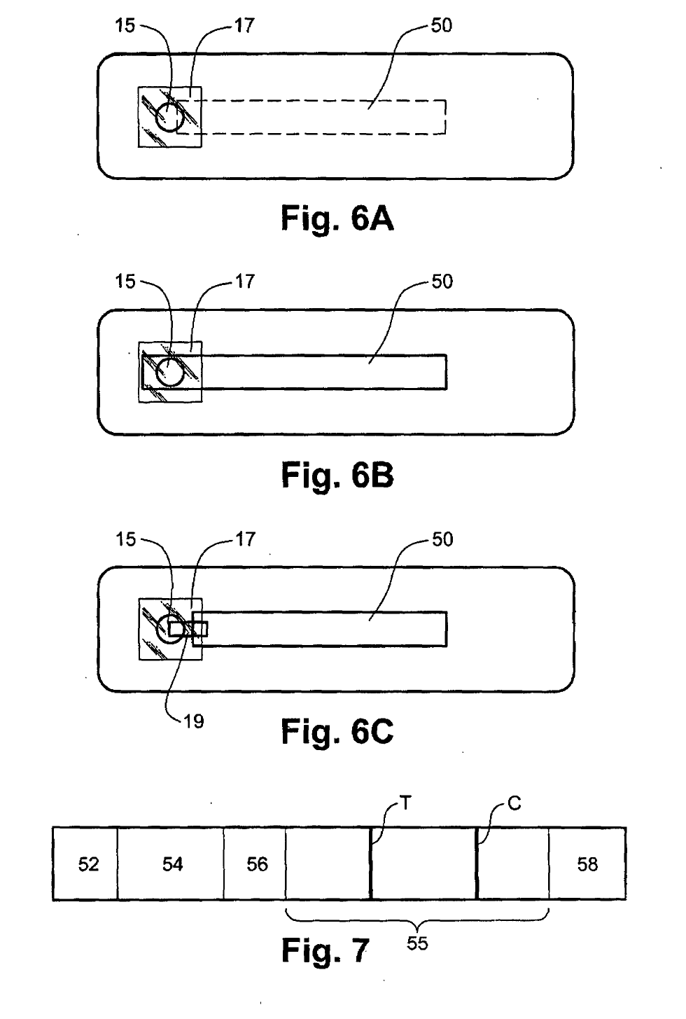

In accordance with the embodiment demonstrated in FIGS. 2 to 6A, the sample receiving zone 52 of strip 50 extends into cavity 16 so that blood entrapped in the cavity is drawn by capillary action to strip 50. However it will be realized that other arrangements are possible which enable flow communication between the blood sample generated in cavity 16 and strip 50. FIGS. 6B and 6C illustrate two such alternatives. In accordance with the embodiment illustrated in FIG. 6B, the sample receiving zone of strip 50 extends over aperture 15 so as to completely cover the skin segment underneath the aperture. Thus, when the lancet is activated, it penetrated the underlying skin through strip 50 to form an immediate direct contact between the strip 50 and the injured area. According to the embodiment in FIG. 6C, a wick member 16, having one end in contact with cavity 16 and a second end in contact with the sample receiving zone of strip 50, serves as a bridging element between the two. Wick member 19 may be made of glass fiber, polyester or other filter material known in the art. For some applications, wick 52 as well as zone 52 may be impregnated with medically approved anticoagulants or bleeding enhancers, such as for example citrate and EDTA.

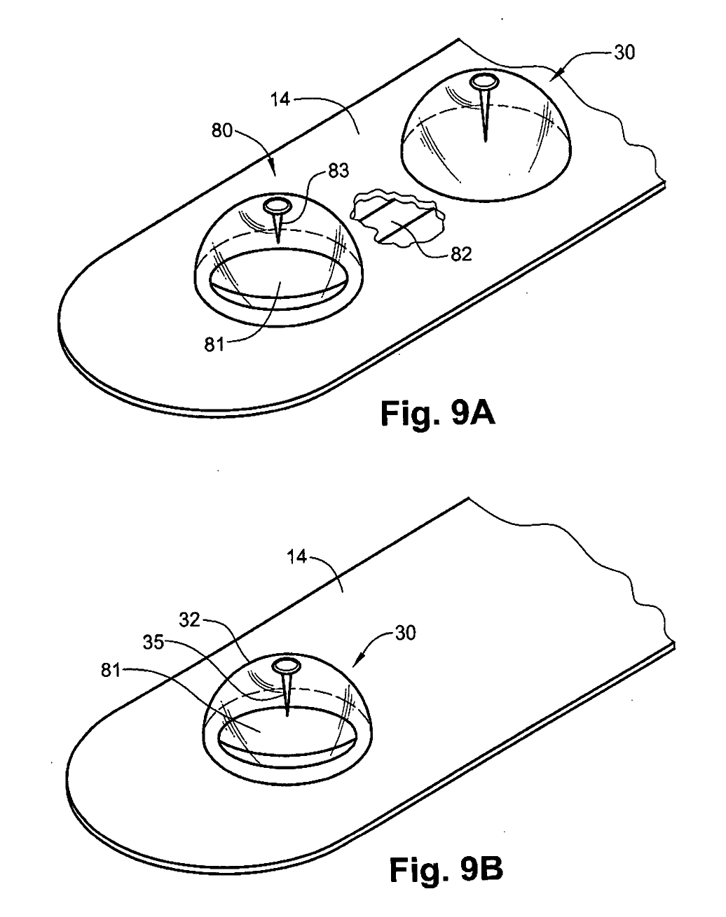

As mentioned above, the size and shape of lancing element 35 as well as the depth to which it penetrates the skin tissue may is designed in accordance with the amount of blood required for the specific diagnostic test strip embedded within the patch. Typically, the amount of blood required for lateral flow assays is in the range of 5 to 100 μL. Such amounts can be easily obtained by means of piercing the top layers of the skin. However it is sometimes necessary to add a small amount of an additional reagent, usually a diluent fluid, such as a buffer solution, in order to perform the test. The buffer could be for example a phosphate buffered saline or Tris buffered saline. For this purpose, the patch of the invention may further include a small reservoir of medically approved buffer or other appropriate reagent solution, adapted to release its content at the same time, or at a predetermined time before or after, the puncturing unit 30 is fired. FIGS. 9A and 9B illustrate two embodiments of the patch of the invention with an additional reagent reservoir 81. In accordance with the embodiment depicted in FIG. 9A, an additional puncturing unit 80, similar in design to puncturing unit 30 but with a shorter lancing element 83, is located downstream of puncturing unit 30. Unit 80 accommodates a blister 81 filled with the required amount of reagent solution, typically in the range of 20 to 100 μL, depending on the specific diagnostic test. Blister 81 may be fabricated from any thin liquid impermeable membrane such as polyethylene, nylon or the like that is easily punctured by a sharp element. Unit 80 is located upstream to and in flow communication with channel 82 (shown in FIG. 9A through a tear in layer 14) that connects reservoir 80 and cavity 16. Channel 82 is formed by an opening in layer 17 which according to this configuration extend over the area below unit 80 and is limited between layers 12 and 14 such that reagent solution released from blister 81 flows into cavity 16. In accordance with this embodiment, blister 81 is punctured preferably either simultaneously or shortly after unit 30 is fired such that the reagent solution released from blister 81 carries the blood as it flows into cavity 16 and further into strip 50. FIG. 9B depicts another configuration according to which blister 81 is located inside puncturing unit 30 on top of opening 15. In accordance with this embodiment, upon firing unit 30, lancet 32 will puncture first the blister and then the skin, enabling drawing blood and releasing the buffer at the same action.

Diagnostic strip 50 may be any diagnostic test strip known in the art for detecting an analyte in a whole blood sample by a lateral flow assay, including immunoassays, enzymatic assays, biochemical assays and chemical assays. FIG. 7 illustrates a typical test strip suitable for use in the present invention, comprising a sample receiving zone 52, a whole blood separation zone 54, a reaction zone 56, a detection zone 55 and an absorbent pad or wick 58 for receiving the fluid and promoting capillary flow through the strip. The different zones may be constructed from one or more bibulous or non-bibulous porous solid phase materials ordered sequentially in an abutting or partial overlapping manner to form a fluid communication therebetween. Strip 50 may be supported on a backing support and/or laminated between two impermeable non-absorbing films such as mylar films, at least one of which is transparent or translucent for allowing viewing the signal. The lateral flow assay is carried out by applying the sample at the sample receiving zone 52 and allowing it to travel along the strip by capillary action, to react with the reagents provided in zone 56 and further downstream to be captured and concentrated at the detection/capture zone 55. Sample receiving zone 52 is the area of a test strip 50 where the sample is applied. Sample receiving zone 52 can include a bibulous or non-bibulous material, such as filter paper, nitrocellulose, glass fibers, polyester or other appropriate materials. Zone 52 can also include compounds or molecules that may be necessary or desirable for optimal performance of the test, for example, buffers, stabilizers, surfactants and the like. Separation zone 54 is constructed from a material capable of separating the fluid portion of the whole blood sample from the red blood cells by entrapping and retaining the red blood cells therein while transporting the blood plasma or blood serum downstream along the strip so as not to obscure the detection zone 55 by the red color of the red-blood cells. Separation zone 54 may be made of a porous membrane that acts as a physical barrier for the red blood cells or may be treated with cell agglutinating reagent to facilitate the separation of the red blood cells from the blood fluid. Reaction or reagent zone 56 is where reagents useful in the detection of the analyte, such as a labeled specific binding member of a first specific binding pair, are bound either movably or immobilized. Typically, the analyte, when present, reacts with the reagents impregnated in zone 56 to form signal generating products that are carried further to be caught at detection zone 55. Detection zone 55 typically comprises a region T where a member of second specific binding pair, different from the first binding specific pair, is immobilized to the strip for capturing the analyte-label pair thereby producing a signal. Detection zone 55 may further includes a control zone C to indicate that the test on the test has performed correctly. It will be realized that FIG. 7 is given by way of illustration only and that other test strips of different structures may be used without departing from the scope of the present invention.

It will be appreciated by persons skilled in the art that the present invention is not limited to what has been particularly shown and described hereinabove. Rather the scope of the present invention is defined only by the claims which follow.

1. A diagnostic device for performing a diagnostic blood test of a subject comprising a flexible patch attachable to the subject's skin, a puncturing unit for producing a blood sample of the subject, and an at least one test element embedded in said patch in a flow communication with said blood sample.

2. The diagnostic device of claim 1 wherein the device comprises a display window through which test results can be read.

3. The diagnostic device of claim 2 wherein said test results are visibly read.

4. The diagnostic device of claim 1 wherein said patch comprises a first flexible sheet comprising an aperture located opposite the puncturing unit and a second flexible sheet overlaying said first flexible sheet and wherein said test element is sandwiched between said first and second flexible sheets in a fluid communication with said aperture.

5. The diagnostic device of claim 1 wherein the puncturing unit comprises a lancing element mounted within a flexible housing.

6. The diagnostic device of claim 1 wherein said at least one test element is a diagnostic strip adapted for a lateral flow assay of a whole blood sample.

7. The diagnostic device of claim 5 wherein said assay is an immunoassay, or an enzymatic assay, or a biochemical assay or a chemical assay.

8. The diagnostic device of claim 5 wherein said assay is a positive/negative assay.

9. The diagnostic device of claim 6 wherein said diagnostic strip is configured for detecting the presence and/or concentration of an analyte in a sample applied thereto.

10. The diagnostic strip of claim 9 wherein said analyte is a blood borne pathogen.

11. The device of claim 9 wherein said diagnostic strip is impregnated with one or more reagents disposed along the strip, said reagents are selected to provide a visible signal when said analyte is present in said sample.

12. The diagnostic device of claim 6 wherein said diagnostic strip comprises a whole blood separation zone for entrapping and retaining red blood cells.

13. The diagnostic device of claim 6 wherein said diagnostic strip comprises a sample receiving zone, a reaction zone and a detection zone.

14. The device of claim 1 further comprising a reservoir of a releasable reagent solution adapted to release the reagent solution to facilitate running the diagnostic test.

15. The device according to claim 14 wherein said reservoir of a releasable reagent solution comprises a blister made of liquid impermeable film encapsulating said reagent solution.

16. The device according to claim 15 wherein said blister is accommodated within the puncturing unit.

17. The device according to claim 15 wherein said blister is accommodated within a second puncturing unit located upstream the puncturing unit for producing the blood sample of the subject and wherein the patch comprises a channel connecting between said two puncturing units to form a flow communication between the reagent solution released from said blister and the blood sample.

18. The device of claim 1 further comprising a safety means to prevent premature activation of the puncturing unit.
